(19) United States
(12) Patent Application Publication
(10) Pub. No.: US 2026/0340046 A1
(43) Pub. Date: Oct. 29, 2026

(54) DATA ANALYSIS ASSISTANCE APPARATUS AND DATA ANALYSIS ASSISTANCE METHOD

(71) Applicant: Hitachi, Ltd., Tokyo (JP)

(72) Inventors: Hiroki KATO, Tokyo (JP); Kunihiko KIDO, Tokyo (JP); Yoichi NAKAMOTO, Tokyo (JP)

(21) Appl. No.: 19/324,599

(22) Filed: Sep. 10, 2025

(30) Foreign Application Priority Data
Oct. 31, 2024 (JP) 2024-191653

(51) Int. Cl. G06F 16/906

(52) U.S. Cl. CPC G06F 16/906

(57) ABSTRACT

A data analysis assistance apparatus includes: a feature vector generation unit that generates a feature vector for each sample based on a data group relating to the samples; a model generation unit that generates a linear model that predicts an evaluation value of predetermined circumstances, based on the feature vector of each sample and the evaluation value relating to the predetermined circumstances of a corresponding sample; a clustering unit that performs clustering and generates clusters, based on coefficients of the linear model for each sample; and an inverse analysis unit that performs inverse analysis of the feature vector for each cluster and identifies data corresponding to the feature vector from the data group.

### CROSS-REFERENCE TO PRIOR APPLICATION

This application relates to and claims the benefit of priority from Japanese Patent Application number 2024-191653, filed on Oct. 31, 2024 the entire disclosure of which is incorporated herein by reference.

### BACKGROUND

The present invention generally relates to a technology of a data analysis assistance apparatus and a data analysis assistance method, and for example, relates to a technology that enables assistance of data analysis with favorable accuracy and efficiency in a manner reducible to a validated medical statistical method.

Real-world data (hereinafter, RWD) obtained from various medical institutions, wearable devices, and the like is utilized for various purposes, such as the development of new drugs and clinical trials by pharmaceutical companies, or for the verification of treatment efficacy in clinical settings. Thus, the RWD has been analyzed to estimate the correlation between specific circumstances (e.g., attributes of patients, diseases, treatment methods, administered drugs, and the like, and drug administration methods) and the desired prediction target circumstances (e.g., prognosis progress, probability of occurrence of side effects and complications, treatment efficacy).

On the other hand, with the advancement of information processing technology, communication technology, and the like in recent years, it has become possible to efficiently acquire the RWD, and there is a tendency for the amount of data subject to the above analysis to become enormous. In this case, analysis adopting a nonlinear model such as machine learning is preferable from the perspective of analysis result accuracy, analysis efficiency, and the like. However, when constructing such a model, it is necessary to select individual features from time-series data indicated by the RWD. In this case, information relating to time-series and relational characteristics of the data is lost, which more easily adversely affects the accuracy of the constructed model. In addition, the selection of the above features requires a high level of expertise and is time-consuming.

Accordingly, a technology disclosed in Japanese Translation of PCT International Application Publication No. 2024-500182 has been proposed as a conventional technology related to the above problems. In this Japanese Translation of PCT International Application Publication No. 2024-500182, the technology relating to a method, a system, and an apparatus for an explainable transducer and an explainable transformer is disclosed. The above technology relates to an explainable machine learning system including a finite state transducer (FST) configured to enable the machine learning system to translate, map, and convert data from an input language to a second output language. The finite state transducer is adapted to receive the data from the input language including one or more input features used for conversion or transforms the one or more input features. The finite state transducer is configured to provide the translated, mapped, and/or converted data as the second output language based on the one or more input features, and/or includes an explainable transformer having an attention-based architecture based on a combination of decoder and encoder components formed in relation to the data from the input language. The finite state transducer and the explainable transformer are combined to generate an explainable transducer transformer (XTT) configured to translate, map, and/or convert the data from the input language.

In Japanese Patent Laid-Open No. 2023-039884, a technology is disclosed for improving the accuracy of word embedding in natural language processing. The aforementioned technology relates to a medical information processing apparatus including a storage unit that stores parameters relating to similarity of semantic relationships between a plurality of medical terms, and a processing circuit that trains a model including a vector representation of each of the plurality of medical terms based on the parameters. The training of the model includes at least one training task in which the model is trained using the parameters, and a further different training task in which the model is trained using word context within a text corpus.

### SUMMARY

In the above conventional technologies, foundation model is constructed by training a transformer-based model using learning data specialized in the medical domain. This foundation model has the advantage of not requiring data selection, since it is possible to directly receive the RWD and embed the RWD into a vector space. It is possible to generate the features while retaining information about the time-series and relational characteristics. In other words, according to the above conventional technologies, it is possible to construct a prediction model without the need of data selection while retaining the information about the time-series characteristics and the like.

However, an analysis result of the RWD often requires explicit demonstration of its validity from the perspective of evidence submission to public institutions and the like. In this regard, since both the foundation model in the above conventional technologies and the subsequent prediction model are nonlinear models, the analysis results thereof are black-boxed, making it difficult to revert to validated traditional medical statistics. In other words, in the analysis of the RWD, it remains difficult to adopt a method employing a nonlinear model.

Accordingly, the present invention has been devised in view of the above problems, and has an object to provide a technology that enables assistance of data analysis with favorable accuracy and efficiency in a manner reducible to a validated medical statistical method.

A data analysis assistance apparatus according to an aspect of the present invention includes: a feature vector generation unit configured to generate a feature vector for each of a plurality of samples, based on a data group relating to the plurality of samples; a model generation unit configured to generate a linear model that predicts an evaluation value of predetermined circumstances, based on the feature vector of each of the plurality of samples and the evaluation value relating to the predetermined circumstances of a corresponding one of the plurality of the samples; a clustering unit configured to perform clustering of the plurality of samples and generate clusters, based on coefficients of the linear model for each of the plurality of samples; and an inverse analysis unit configured to perform inverse analysis of the feature vector for each of the generated clusters and identify data corresponding to the feature vector from the data group relating to the plurality of samples.

According to the present invention, assistance is possible of data analysis with favorable accuracy and efficiency in a manner reducible to a validated medical statistical method.

### BRIEF DESCRIPTION OF THE DRAWINGS

FIG. 1 is a diagram showing a network configuration including a data analysis assistance apparatus according to an embodiment.

FIG. 2 is a diagram showing a hardware configuration example of the data analysis assistance apparatus according to the embodiment.

FIG. 3 is a diagram showing a function configuration example of the data analysis assistance apparatus according to the embodiment.

FIG. 4 is a diagram showing a concept and the like of a foundation model and an explainable AI according to the embodiment.

FIG. 5 is a diagram showing a concept example of clustering processing according to the embodiment.

FIG. 6 is a diagram showing a concept example of inverse analysis processing according to the embodiment.

FIG. 7 is a diagram showing a configuration example of RWD according to the embodiment.

FIG. 8 is a diagram showing a processing flow example of a data analysis assistance method according to the embodiment.

FIG. 9 is an explanatory diagram showing a rearrangement example of feature vectors.

FIG. 10 is an explanatory diagram showing a structure example of a neural network.

FIG. 11 is a block diagram showing a functional configuration example of part of the data analysis assistance apparatus.

### DETAILED DESCRIPTION

In the following description, “CPU” is an arithmetic apparatus and may be one or more processor devices. At least one processor device may typically be microprocessor device such as a central processing unit (CPU), but may also be a processor device of another type such as a graphics processing unit (GPU). At least one processor device may be single-core or may be multi-core. At least one processor device may be a processor core. At least one processor device may be a processor device in broad terms, such as a hardware circuit (for example, a field-programmable gate array (FPGA)) that performs a partial or an entirety of processing, a complex programmable logic device (CPLD), or an application-specific integrated circuit (ASIC).

In the following description, “storage apparatus” may be one or more permanent memory devices being examples of one or more storage devices. The permanent storage device may typically be non-volatile storage device. In particular, the permanent storage device may, for example, be a hard disk drive (HDD), a solid-state drive (SSD), or a non-volatile memory express (NVMe) drive.

In the following description, “memory” may be one or more memory devices being examples of one or more storage devices. At least one memory device in the memory may be a volatile memory device or may be a non-volatile memory device.

In the following description, “communication apparatus” may be one or more communication interface devices. The one or more communication interface devices may be one or more communication interface devices of the same type (for example, one or more network interface cards (NICs)) or may be communication interface devices or two or more different types (for example, a NIC and a host bus adapter (HBA)).

In the following description, information obtained through output with respect to input may be described using expressions such as “*** table” or “*** database”, but the information may be data of any structure (for example, may be structured data or may be unstructured data), or may be a learning model which is typically a neural network, a genetic algorithm or a random forest that produces output with respect to input. Therefore, “*** table” or “*** database” can also be referred to as “*** information”. In the following description, a configuration of each database or table is one example. One database or table may be divided into two or more databases or tables, or a part or an entirety of two or more databases or tables may be one database or table.

In the following description, a case of processing may be described with “program” as a subject thereof, but the subject of the processing may also be a CPU (or device such as a controller including the processor), since predetermined processing is performed by appropriately using a storage apparatus, an interface apparatus, and/or the like, by executing the program via the CPU. The program may be installed on an apparatus such as a computer from a program source. The program source may be a (for example, non-transitory) recording medium readable by a program distribution server or the computer. In the following description, two or more programs may be implemented as one program or one program may be implemented as two or more programs.

In the following description, when elements of the same type are described without being distinguished from one another, common portions of reference signs may be used. When distinguishing elements of the same type from one another, reference signs or element identifiers may be used.

### Network Configuration of Data Analysis Assistance Apparatus

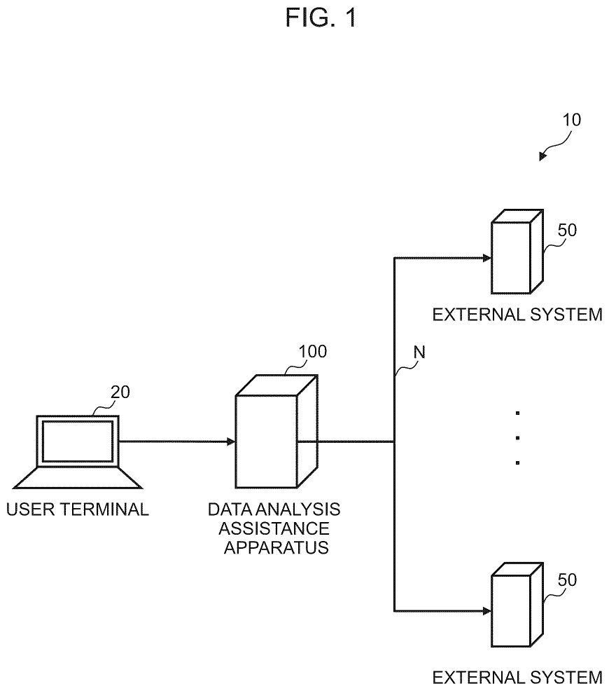

FIG. 1 is a diagram showing a network configuration example of a data analysis assistance apparatus 100 according to an embodiment.

The data analysis assistance apparatus 100 of the present embodiment is an apparatus that enables assistance of data analysis with favorable accuracy and efficiency in a manner reducible to a validated medical statistical method. This data analysis assistance apparatus 100 is connected via an appropriate network N to a user terminal 20 and an external system 50 so as to enable cooperation as necessary. Therefore, the data analysis assistance apparatus 100, the user terminal 20, and the external system 50 may constitute a data analysis assistance system 10.

In the above network configuration, the data analysis assistance apparatus 100 is an information processing apparatus that serves as an executing entity of a data analysis assistance method of the present embodiment. This data analysis assistance apparatus 100 is operated by an entity such as a pharmaceutical company engaged in new drug development or a system integrator that performs assistance thereof. More specifically, the data analysis assistance apparatus 100 is a server apparatus that provides the user terminal 20 with a service (merely an example of the embodiment).

The user terminal 20 is a terminal that accesses the data analysis assistance apparatus 100 via the network N and displays an execution result of the data analysis assistance method of the present embodiment on a screen using a predetermined viewer such as a web browser. This user terminal 20 is a client used by a user such as personnel in a research and development department of the above pharmaceutical company, customer support personnel at the system integrator, or the like. Specifically, the user terminal 20 is configured as, for example, a personal computer (PC), a tablet terminal, or a smartphone.

The external system 50 is, for example, a system that acquires and manages RWD at a medical institution. The external system 50 receives a request from the data analysis assistance apparatus 100 or the user terminal 20 and responds with the RWD at fixed intervals. Note that the RWD is various types of medical data, which may include medical claims data, diagnosis procedure combination (DPC) data, electronic medical record data, health examination data, patient registry data, and biological data obtained from a wearable device.

### Hardware Configuration of Data Analysis Assistance Apparatus

Next, a hardware configuration of the above data analysis assistance apparatus 100 will be described.

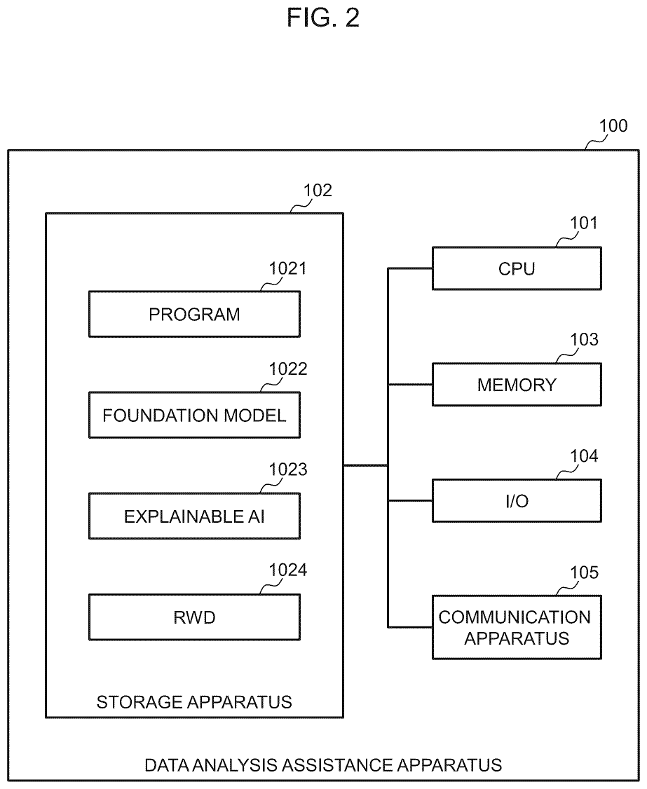

FIG. 2 is a diagram showing a hardware configuration example of the data analysis assistance apparatus 100 according to the present embodiment.

The data analysis assistance apparatus 100 according to the present embodiment includes a CPU 101, a storage apparatus 102, a memory 103, an I/O 104, and a communication apparatus 105.

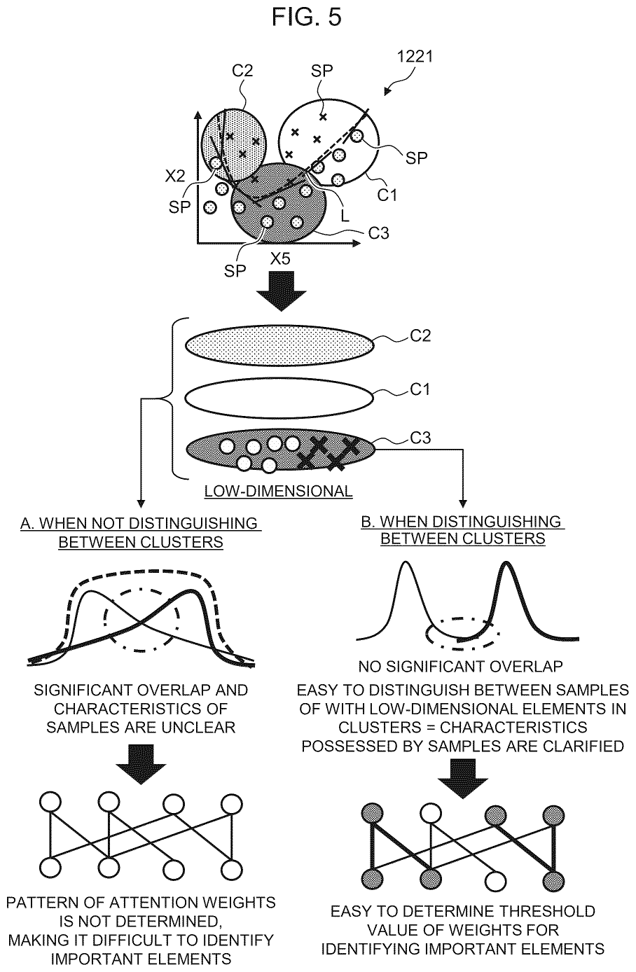

Among these, the CPU 101 is a processor that executes a program 1021 held in the storage apparatus 102 by loading the program 1021 into the memory 103, and performs comprehensive control of the apparatus itself as well as various determination, arithmetic, and control processing. In other words, functional units corresponding to the data analysis assistance method implemented in the data analysis assistance apparatus 100 (see FIG. 5; a feature vector generation unit 120, a model generation unit 121, a clustering unit 122, an inverse analysis unit 123, and an output unit 124) are implemented through the CPU 101 executing the program. Note that part of processing performed through the CPU 101 executing the program 1021 may be executed by another arithmetic apparatus (for example, hardware such as an ASIC or an FPGA). The above functional units (the feature vector generation unit 120, the model generation unit 121, the clustering unit 122, the inverse analysis unit 123, and the output unit 124) will be described in detail below.

The storage apparatus 102 is a storage means configured as a non-volatile storage apparatus such as a hard disk drive or an embedded multimedia card. Note that RWD 1024 is present as data held by the storage apparatus 102. A specific configuration of this RWD 1024 will be described below. The storage apparatus 102 holds a foundation model 1022 and an explainable AI 1023. The foundation model 1022 and the explainable AI 1023 will be described in detail below.

Note that the program 1021 executed by the CPU 101 may be provided to the data analysis assistance apparatus 100 via removable media (CD-ROM, flash memory, or the like) or the network N, and may be stored in the non-volatile storage apparatus 102 being a non-transitory storage medium.

The memory 103 is a storage means configured as a volatile storage apparatus such as random-access memory (RAM). Note that the memory 103 may be read-only memory (ROM) being a non-volatile storage element. The ROM is stored in an immutable program (for example, a BIOS).

The I/O 104 is an interface connected to an input apparatus such as a keyboard and an output apparatus such as a display. Note that the user terminal 20 connected to the data analysis assistance apparatus 100 via the network N may provide the input apparatus and the output apparatus via the above I/O 104.

The communication apparatus 105 is an apparatus that is connected to the network N, and performs communication with the user terminal 20, the external system 50, and the like.

Note that the data analysis assistance apparatus 100 is a computer system physically consisting of one computer, or logically or physically consisting of a plurality of computers. The data analysis assistance apparatus 100 may operate using a virtual computer constructed from resources of a plurality of physical computers. The data analysis assistance apparatus 100 may be implemented on a cloud, or may be on-premises equipment implemented on a specific computer (hardware).

The network N connecting the data analysis assistance apparatus 100 to the user terminal 20, the external system 50, and the like can be assumed to be the Internet, a local area network (LAN), a wide area network (WAN), a mobile telephone network, or the like, but is not limited thereto. Here, a general public network, for example, a 5th generation (so-called 5G) mobile communication system that enables “multiple simultaneous connections” and “ultra-low latency” may be used as an example of a mobile telephone network or the like regardless of the network being wired or wireless. By further leveraging the characteristics of new mobile phone systems beyond 5G, it is possible to anticipate enhancement of secondary effects such as acceleration of processing in the data analysis assistance method according to the invention of the present application and improvement of high-definition rendering of output information.

Note that data transmission and reception between the data analysis assistance apparatus 100, and the user terminal 20, the external system 50, and the like may, for example, be implemented in accordance with an application programming interface (API) protocol. In this case, functions and configurations for executing request and response processing through the API may be implemented in advance in all equipment.

### Foundation Model, Explainable AI, and Functional Units

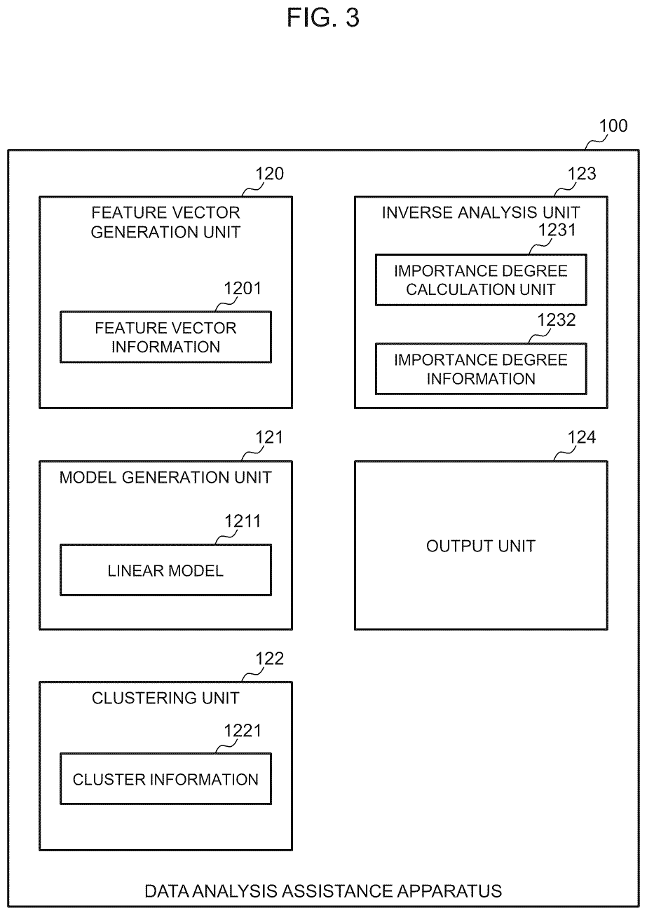

Next, the foundation model 1022, the explainable AI 1023, and functional units will be described with reference to FIGS. 3 to 5.

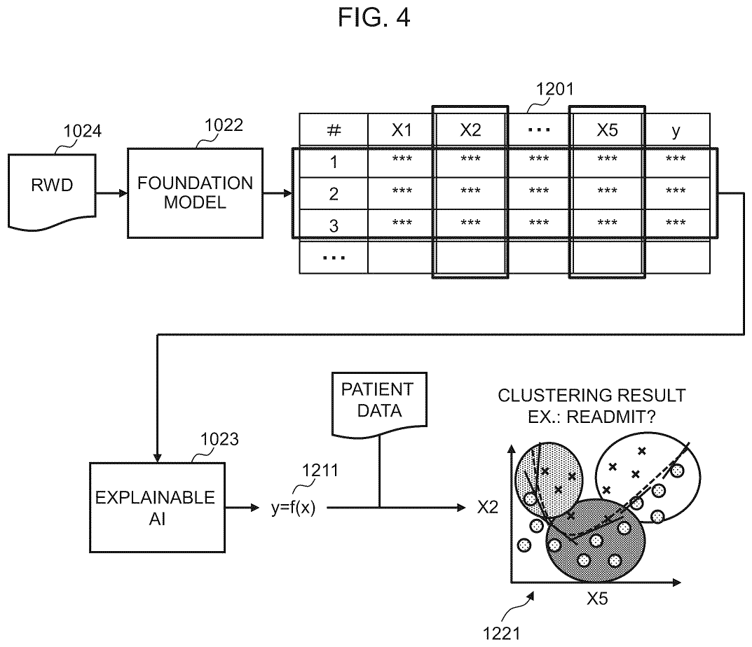

FIG. 3 is a diagram showing a function configuration example of the data analysis assistance apparatus 100 according to the present embodiment. FIG. 4 is a diagram showing a concept and the like of the foundation model 1022 and the explainable AI 1023 according to the present embodiment. FIG. 5 is a diagram showing an example of clustering processing according to the present embodiment.

Among these, the foundation model 1022 is a machine learning model obtained by performing pre-training using a large amount of data. This pre-training is performed using so-called self-supervised learning (SSL; training method using a large dataset without assigning teacher labels).

Note that the foundation model 1022 according to the present embodiment is retrained to be able to execute a specific task related to the RWD 1024 collected from the medical institutions or the like. The above task is executed by the feature vector generation unit 120 according to the present embodiment. For example, n-dimensional feature vectors X1 to X5, that is, feature vector information 1201 is generated for each of a plurality of samples (e.g., patients), based on the RWD 1024 (data group relating to the plurality of samples).

Each of the n-dimensional feature vectors obtained in this way is extracted and generated by the foundation model 1022 based on perspectives and algorithms obtained in prior training and retraining, thus maintaining time-series characteristics, relational characteristics, and the like of the RWD 1024. On the other hand, values relating to these time-series characteristics, relational characteristics, and the like are embedded in a vector space. Thus, it is difficult to interpret the content, meaning, and the like of the feature vectors even when a human attempts to do so. However, as described above, each of the n-dimensional feature vectors is a dataset in which the time-series characteristics and the relational characteristics of the RWD 1024 are maintained, which has the advantage that no information is lost. There is also the advantage that the user does not incur any costs for data selection, feature vector generation, or the like from the RWD 1024.

Upon providing the above feature vector information 1201, the explainable AI 1023 generates a linear model 1211 that predicts an evaluation value of predetermined circumstances of a patient (e.g., treatment prognosis such as a probability of readmission), based on the n-dimensional feature vectors X1 to X5 per patient and an evaluation value (y: probability value of the above prognosis) relating to the predetermined circumstances. Such an explainable AI 1023 is the model generation unit 121.

The explainable AI 1023 according to the present embodiment has, for example, a function of searching for critical factors that influence the effects of pharmaceuticals (biomarkers: characteristics objectively measured and evaluated as indices of disease diagnosis, treatment effects, and pharmaceutical effects). The explainable AI 1023 can present information relating to supporting proof and particulars of a derived answer, and has effects valid for decision-making within medical and drug discovery fields. In particular, it is possible to search for a new factor that combines a variable and an operator, for example, a new factor making it possible to accurately predict an outcome such as BMI=weight÷height². This contributes to the generation of the linear model 1211 that predicts the evaluation value (y: probability value of the above prognosis) relating to treatment prognosis such as the probability of readmission in the present embodiment.

The explainable AI 1023 may be based on a technology of the present applicant (see Japanese Patent Laid-Open No. 2020-194414) for extracting an important feature from non-obvious features that contribute to prediction. The explainable AI 1023 based on the above technology executes selection processing of selecting a first feature group, which self-evidently contributes to the prediction of the predetermined circumstances, and a second feature group other than the first feature group from among a set of features, that is, the n-dimensional feature vectors.

In a loss function relating to a difference between a prediction result output when the above n-dimensional feature vectors are input to the prediction model and correct data corresponding to the features, the explainable AI 1023 executes manipulation processing of manipulating a first regularization coefficient relating to a first weight parameter group corresponding to the first feature group such that the loss function increases and manipulating a second regularization coefficient relating to a second weight parameter group corresponding to the second feature group such that the loss function decreases, the first weight parameter group and the second weight parameter group each being part of a weight parameter set constituting the prediction model.

The explainable AI 1023 executes learning processing of learning the weight parameter set of the prediction model such that the loss function is minimized, as a result of manipulating the first regularization coefficient and the second regularization coefficient in the above manipulation processing. The explainable AI 1023 executes importance degree calculation processing (processing by an importance degree calculation unit 1231) that calculates an importance degree of the features based on the above weight parameter set. In this case, the explainable AI 1023 selects, in the above selection processing, the first feature group and the second feature group based on the importance degree calculated in the importance degree calculation processing. For example, in the example shown in FIG. 4, the first feature group and the second feature group described above are selected as the feature vectors X5 and X2. A function and a configuration of such an explainable AI 1023 will be described below, but the present explainable AI is a linear model that predicts the evaluation value of the predetermined circumstances from the n-dimensional feature vectors X1 to X5 per patient. Specifically, the parameters are learned so that a linear prediction model can be obtained while rearranging the n-dimensional features X1 to X5 for each patient from a certain feature space to another feature space. As a result, a linear model can be obtained that predicts the evaluation value of the predetermined circumstances using a coefficient of a linear sum of the n-dimensional features X1 to X5 per patient in a feature space in which the evaluation value can be classified using the linear prediction model. Since the present explainable AI is linear, it is possible to understand weighting of the feature vectors when referring to a slope of the explainable AI. When executing post-processing of local linear approximation after obtaining a nonlinear prediction model, it is uncertain whether a local portion of the linearly approximated prediction model can accurately explain the feature vectors, but it is possible to correctly explain the feature vectors since the present explainable AI derives a linear model. That is, since the present explainable AI understands the weighting of the feature vectors based on the linear model in which the feature vectors can be correctly explained, it is possible to perform dimensionality reduction of the vectors without the feature groups selected based on the importance degree calculated in the importance degree calculation processing losing important features. Performing the dimensionality reduction of the vectors in this way is also valid when clustering. That is, when clustering using multidimensional vectors, it is possible that the multidimensional vectors cannot be categorized into appropriate clusters, but it is more likely that semantically similar clusters (for example, patient group with similar prognoses) can be generated through clustering with important low-dimensional vectors.

The linear model 1211 obtained for each patient in this way is provided to the clustering unit 122. The clustering unit 122 executes clustering of the patient group (see FIG. 5) based on the coefficients of the linear model 1211 for each of the above patients and generates, for example, each cluster 1221 (cluster information), namely clusters C1 to C3. The above coefficients are, for example, values of a slope of a line segment L when the clustering unit 122 executes local linear approximation in a corresponding cluster regarding the linear model 1211 generated for each sample SP (patient) of each cluster C1 to C3. Processing details in such a clustering unit 122 involve generating a cluster (patient group) with similar feature vectors by performing k-means clustering on importance degree and relocation vectors, as indicated by the technology developed by the present applicant (see Japanese Patent Laid-Open No. 2023-036144). Note that the clustering processing details may also be based on content described in the above publication (Japanese Patent Laid-Open No. 2023-036144), but detailed description thereof is omitted here.

The inverse analysis unit 123 performs inverse analysis of the feature vectors per cluster generated by the clustering unit 122, and identifies data corresponding to the feature vectors from a data group relating to the plurality of samples. The inverse analysis unit 123 calculates the importance degree of the feature vectors in the above linear model 1211 using the importance degree calculation unit 1231, and performs inverse analysis on feature vectors with an importance degree equal to or larger than a threshold value.

In the examples of FIGS. 4 to 6, the feature vectors with an importance degree equal to or larger than the threshold value are the feature vectors X2 and X5. In this case, the inverse analysis unit 123 performs narrowing down of similar patients (for example, various attributes are identical/similar) to the patients being a generation target of the linear model 1211, based on the values of the feature vectors X2 and X5, and executes inverse analysis using the foundation model 1022 based thereon. As a result, target data is identified from the RWD 1024.

As shown in FIG. 5, when observing distribution circumstances of the samples per cluster (B. when distinguishing between clusters), a function representing the distribution of the samples in each cluster is low-dimensional (for example, one-dimensional), clarifying characteristics possessed by the samples. On the other hand, when observing the distribution circumstances of the samples without distinguishing between the clusters (A. when not distinguishing between clusters), a function representing the distribution of the samples in all clusters exhibits significant overlap even when low-dimensional, obfuscating the characteristics possessed by the samples. Thus, when performing inverse analysis using the inverse analysis unit 123, cost of attention weight calculation in the foundation model 1022 is reduced by distinguishing between clusters, that is, by clarifying the clusters. Data with attention weights equal to or larger than a threshold value is identified in the data group relating to the samples, that is, the RWD 1024, but when observing the distribution circumstances of the samples without distinguishing between the clusters (A. when not distinguishing between clusters), a pattern of the attention weights is not determined, making it difficult to identify important elements. On the other hand (B. when distinguishing between clusters), it is easy to determine the threshold value of the weights for identifying the important elements, since the characteristics possessed by the samples are clarified. By conducting inverse analysis per patient group cluster based on the coefficients of the linear model 1211 for each patient in this way, it becomes easier to identify data based on the attention weights of the foundation model 1022, in other words, it is possible to facilitate identification of data with important meaning from the RWD 1024. For example, identifying information from the RWD that influences treatment prognosis becomes easier.

The output unit 124 distributes the information about the data identified by the above inverse analysis unit 123 to the user terminal 20 and performs output control such as display.

### RWD

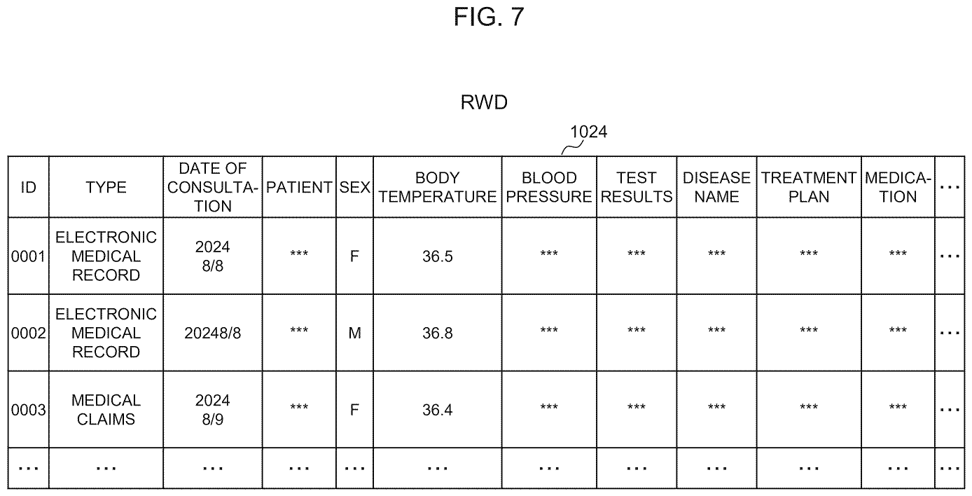

Next, various types of data held by the data analysis assistance apparatus 100 in the storage apparatus 102 will be specifically described. FIG. 7 is a diagram showing a configuration of the RWD 1024 according to the present embodiment. The RWD 1024 is, for example, medical data collected from the medical institutions or the like, and corresponds to medical claims data, diagnosis procedure combination (DPC) data, electronic medical record data, health examination data, patient registry data, and biological data obtained from a wearable device, as described above.

In the example of the RWD 1024 shown in FIG. 7, the RWD 1024 is an aggregate of records in which an ID uniquely identifying each record is associated with values such as type of data, date of consultation, patient (classification value), sex, body temperature, blood pressure, test results, disease name, treatment plan, and medication.

### Data Analysis Assistance Method: Processing Flow

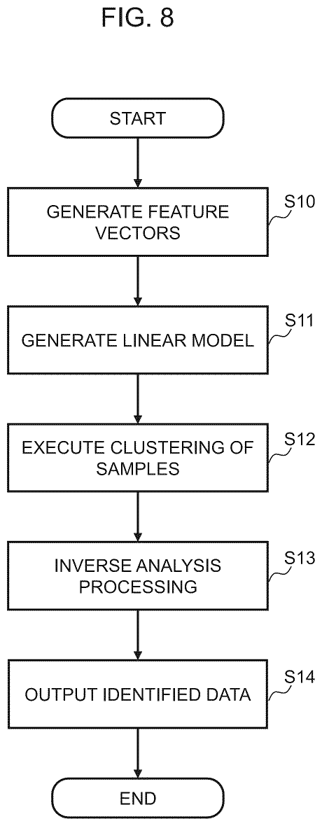

Next, a processing flow of the data analysis assistance method according to the present embodiment will be described. FIG. 8 is a diagram showing a processing flow example of the data analysis assistance method according to the present embodiment. First, the feature vector generation unit 120 generates, for example, the n-dimensional feature vectors X1 to X5, that is, the feature vector information 1201 for each of the plurality of samples (e.g., patients), by providing the RWD 1024 (data group relating to the plurality of samples) to the foundation model 1022 (S10).

Each of the n-dimensional feature vectors obtained in this way is extracted and generated by the foundation model 1022 based on perspectives and algorithms obtained in prior training and retraining, thus maintaining time-series characteristics, relational characteristics, and the like of the RWD 1024. When the processing is executed by the feature vector generation unit 120 as described above, the user does not incur any costs for data selection, feature vector generation, or the like from the RWD 1024.

Next, the model generation unit 121 provides the feature vector information 1201 obtained in the above S10 to the explainable AI 1023, and generates the linear model 1211 that predicts the evaluation value of the above predetermined circumstances of the patient (e.g., treatment prognosis such as the probability of readmission), based on the n-dimensional feature vectors per patient and the evaluation value (y: probability value of the above prognosis) relating to the predetermined circumstances (S11).

The linear model 1211 obtained for each patient in the above S11 is provided to the clustering unit 122. In this case, the clustering unit 122 executes clustering of the patient group (see FIG. 5) based on the coefficients of the linear model 1211 for each of the above patients and generates, for example, each cluster 1221 (cluster information), namely clusters C1 to C3 (S12). The above coefficients are, for example, the values of the slope of the line segment L when the clustering unit 122 executes local linear approximation in the corresponding cluster regarding the linear model 1211 generated for each sample SP (patient) of each cluster C1 to C3.

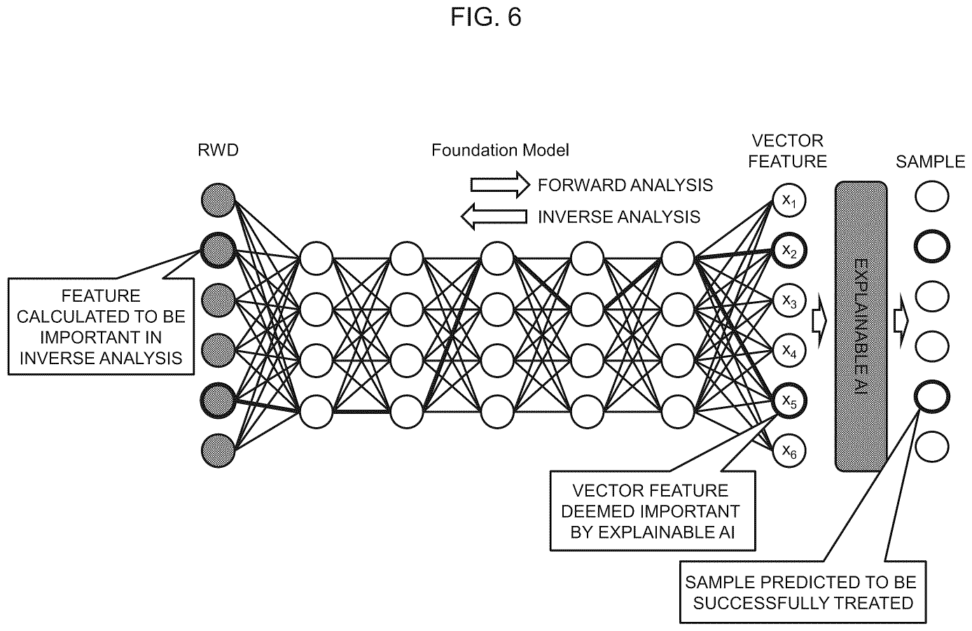

Next, the inverse analysis unit 123 performs inverse analysis of the feature vectors per cluster generated by the clustering unit 122 in the above S12, and identifies the data corresponding to the feature vectors from the data group relating to the plurality of samples, that is, the RWD 1024 (S13). FIG. 6 shows an example of identifying the data corresponding to the feature vectors from the data group relating to the plurality of samples, that is, the RWD 1024. In FIG. 6, inverse analysis of the feature vectors X2 and X5 is performed on samples belonging to the same cluster generated by the clustering unit 122 using samples predicted to be successfully treated, and a data group calculated to be important in the inverse analysis is identified. Deep learning shown as the foundation model may be used, or a model such as a transformer may be used. The inverse analysis unit 123 calculates the importance degree of the feature vectors in the above linear model 1211 using the importance degree calculation unit 1231, and performs inverse analysis on the feature vectors with the importance degree equal to or larger than the threshold value.

The output unit 124 distributes, to the user terminal 20, the data identified by the above inverse analysis unit 123, that is, medical data relating to a corresponding patient among the RWD 1024 (S14), and ends the processing flow.

As described above, according to the data analysis assistance apparatus in the present embodiment, important (semantically similar) data can be obtained by performing inverse analysis in stratified units. That is, assistance is possible of data analysis with favorable accuracy and efficiency in a manner reducible to a validated medical statistical method.

Note that the present invention is not limited to the embodiment described above, and includes various modifications and equivalent configurations within the spirit of the accompanied claims. For example, the embodiment described above has been described in detail for easy understanding of the present invention, and the present invention is not necessarily limited to including all of the configurations described above. Part of the configuration of a certain embodiment can be replaced with the configuration of another embodiment. The configuration of another embodiment can be added to the configuration of the certain embodiment. Part of the configuration of each embodiment may be subjected to addition, deletion and replacement with another configuration.

Some or all of the above-described configurations, functions, processing units, processing means, and the like may be implemented in hardware by designing, for example, an integrated circuit, or may be implemented in software by a processor interpreting and executing a program that implements respective functions.

Information such as a program, a table, and a file that implements each function can be stored in a storage apparatus such as a memory, a hard disk, and a solid-state drive (SSD), or a recording medium such as an Integrated Circuit (IC) card, an SD card, and a digital versatile disc (DVD). Some or all of the above configurations, functions, processing units, processing means, and the like may be implemented as hardware by designing, for example, an integrated circuit. The above configurations, functions, and the like may be implemented as software by a processor interpreting and executing a program that implements respective functions. Information such as a program, a table, and a file that implements each function can be stored in a recording apparatus such as a memory, a hard disk, and a solid-state drive (SSD), or a recording medium such as an Integrated Circuit (IC) card, an SD card, and a digital versatile disc (DVD).

Control lines and information lines indicate what is considered necessary for explanation, and not all control lines and information lines necessary for implementation in the products are shown. It may be considered that almost all the configurations are actually connected to each other.

The various above description can be summarized as follows. The following summary may include supplementary description of the above, description of modified examples, or the like.

The data analysis assistance apparatus of the present embodiment may further include an importance degree calculation unit that calculates an importance degree of the feature vector in the linear model, in which the inverse analysis unit may perform inverse analysis of the feature vector with the importance degree equal to or larger than a threshold value.

According to this, it is possible to obtain important (semantically similar) data, by narrowing down feature vectors with a high importance degree and performing inverse analysis thereof. Thus, assistance is possible of data analysis with favorable accuracy and efficiency in a manner reducible to a validated medical statistical method.

In the data analysis assistance apparatus of the present embodiment, the data relating to the plurality of samples may be data relating to a medicine.

According to this, important (semantically similar) data can be obtained by performing inverse analysis, since original data patterns (diagnostic patterns, time-series patterns, and the like) are limited. Thus, assistance is possible of data analysis with favorable accuracy and efficiency in a manner reducible to a validated medical statistical method.

The data analysis assistance apparatus of the present embodiment may further include an output unit configured to output information about the data corresponding to the identified feature vector.

According to this, it is possible to explicitly present the user with data important for data analysis. Thus, assistance is possible of data analysis with favorable accuracy and efficiency in a manner reducible to a validated medical statistical method.

Hereinafter, a calculation example of the importance degree will be supplemented. Note that in the following supplementary description, a feature vector Xn is denoted as “feature vector xₙ”. The following supplementary description adheres to the description of Example 5 in Japanese Patent Laid-Open No. 2020-194414.

### Rearrangement Example of Feature Vectors

Artificial intelligence (AI) has the capability to solve problems that are not linearly separable, but it is unclear why the AI makes such determinations. In particular, a machine learning method such as deep learning has high prediction accuracy but low explanation capability. For example, when the AI outputs a diagnosis result such as “prone to catching colds” for a certain patient, a physician cannot answer why the AI obtained such a result. If the AI can determine the cause, the physician can administer appropriate treatment to the patient.

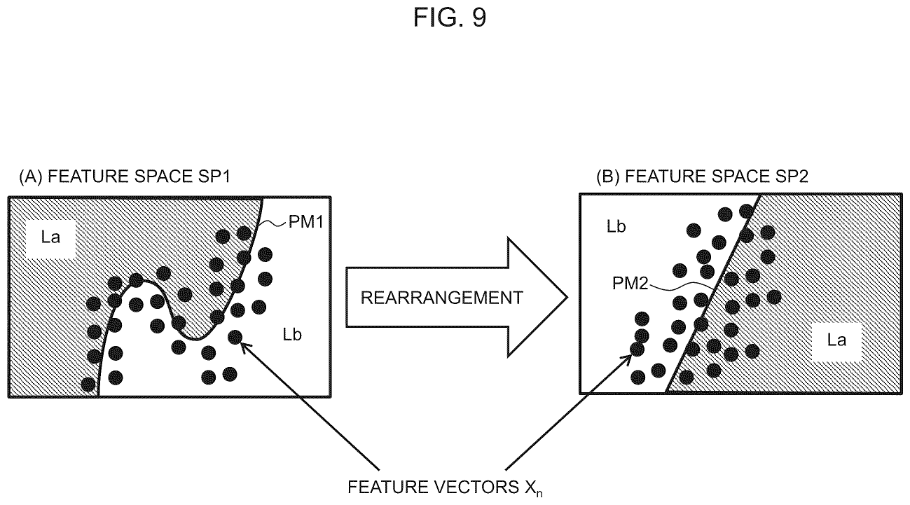

FIG. 9 is an explanatory diagram showing a rearrangement example of the feature vectors. (A) The plurality of feature vectors xₙ (n=1, 2, . . . , N; N is the number of images) are present in a feature space SP1. The plurality of feature vectors xₙ are, for example, classified into correct labels La and Lb by a nonlinear prediction model PM1. (B) The plurality of feature vectors xₙ are present in a feature space SP2. The plurality of feature vectors xₙ are, for example, classified into the correct labels La and Lb by a linear prediction model PM2.
(A) In a machine learning method such as deep learning, a new linear regression is learned in order to explain the nonlinear prediction model PM1 being a classification result. Specifically, in this machine learning method, post-processing is executed of local linear approximation after obtaining the prediction model PM1, for example. However, it is uncertain whether a local portion of the linearly approximated prediction model PM1 can accurately explain the feature vectors xₙ in such post-processing. More importantly, it is necessary to execute machine learning twice in total in order to execute logistic regression being linear approximation.

Since the linear prediction model PM2 of (B) is linear, it is possible to know which parameters in the feature space SP2 the feature vectors xₙ are weighted by and to correctly explain the feature vectors xₙ by referring to a slope of the prediction model PM2. In example 1, the plurality of feature vectors xₙ in the feature space SP1 are rearranged to the other feature space SP2 without obtained the nonlinear prediction model PM1 as in (A). With this, it is possible to know which parameters in the feature space SP2 the feature vectors xₙ are weighted by and to correctly explain the feature vectors xₙ in accordance with the importance degree thereof, since the linear prediction model PM2 can be obtained.

That is, since the user understands which factors (features) contained in the features xₙ contribute to the prediction result per sample (for example, per patient) with the feature vectors xₙ, it becomes easier to explain why such a prediction result is obtained. Therefore, it is possible improve the explanation capability in the machine learning. In the above example, it is possible to know why the AI outputs a diagnosis result such as “prone to catching colds” for a certain patient (for example, because the patient has lost weight). Since it is not necessary to execute machine learning twice as in (A), it is possible to improve the efficiency of the machine learning. Therefore, it is possible to promptly provide the explanation as described above.

### Structure Example of Neural Network

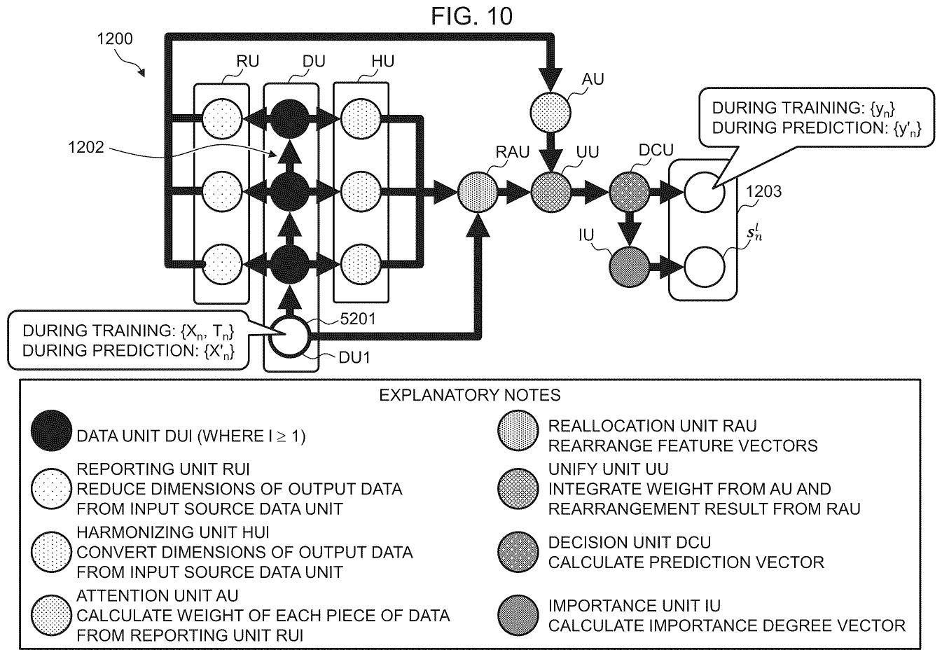

FIG. 10 is an explanatory diagram showing a structure example of a neural network. A neural network 1200 may be a specific example of the explainable AI 1023. The neural network 1200 includes a data unit group DU, a reporting unit group RU, a harmonizing unit group HU, a reallocation unit RAU, a unify unit UU, a decision unit DCU, and an importance unit IU.

The data unit group DU has a configuration in which a plurality of data units DUL (1 is a hierarchical number, 1≤l≤L; L is the lowest-level hierarchical number; L=4 in FIG. 10) is serially connected. The data unit DU (l=1 being a top level) is an input layer 5201 of the neural network 1200, and the data unit DUl (l≤2) corresponds to an intermediate layer (also referred to as a hidden layer) of the neural network 1200. The data unit DUl is a perceptron that inputs output data from the preceding data unit DU (l-1), computes using its own training parameters, and outputs output data.

However, the data unit DUL holds training data during training by a training unit 412. Here, the training data is, for example, sample data including a combination {xₙ, tₙ} of an image being an example of the feature vectors xₙ and its true correct label tₙ (n=1, 2, . . . , N; N is the number of images). An image xₙ is data with a two-dimensional matrix structure, and hereinafter, will be treated as a d-dimensional vector (integer satisfying d≥1) obtained by raster scanning. For ease of description, when denoted as “x”, this refers to a one-dimensional vector obtained by raster scanning the image xₙ in a matrix format.

A correct label tₙ is a K-dimensional vector that represents a category (for example, an animal such as a dog or a cat) using a one-hot expression with respect to a type number K of the image xₙ. In the one-hot expression, a certain element of the vector corresponds to the type of the image xₙ, 1.0 is stored in only one element, and all other elements are 0.0. The type (for example, dog) corresponding to the element 1.0 is the correct type. When a medical image Xₙ such as an X-ray, CT, MRI, or ultrasound is used as input, the label tₙ is a true value representing the type of disease, the prognosis (good or poor) of the patient, or the like.

An image xₙ ∈R^{d} (R^{d} is a d-dimensional real number) is a feature vector consisting of d-dimensional real numbers R^{d}. A function h^{l+1}D indicating a data unit DU (l+1) is expressed with the following expression (1).

$$is an input / output vector of the data unit \qquad [ Math 1 ]$$

$$Where , h is an input / output vector of the data unit \text{?}$$

$${h}_{D}^{l} ∈ {ℛ}^{{d}^{l}} \text{?} is a training parameter \text{?}$$

$$When l = 1 , {d}^{l} \text{?}$$

$$l = 1 \text{?} , {h}_{D}^{1} = {x}_{n}$$

In the expression (1), superscript 1 (integer satisfying 1≤l≤L) indicates a hierarchical number (the same applies to the following expressions). L is an integer of 1 or more, and indicates the deepest-level hierarchical number. f_{D}^{l} on the right-hand side is an activation function. Various activation functions may be used as the activation function, such as a sigmoid function, a hyperbolic tangent function (tanh function), or a rectified linear unit (ReLU) function. A matrix W^{l}_{D} is a training parameter of the data unit DUl. A vector h^{l}_{D} on the right-hand side is an input vector that is input to the data unit DUl, that is, an output vector from the preceding data unit DUl. When the number of layers l=1, an output vector h^{l}_{D} from the data unit DUl is h^{l}_{D}=xₙ.

The data unit DUl holds the image xₙ being a feature vector as test data during prediction in a prediction unit 414.

A reporting unit RUI (2≤l≤L) receives input of the output vector h^{l}_{D} from the data unit DUl in the same layer and reduces the number of dimensions of the output vector h^{l}_{D}. A function h^{l}_{R} indicating the reporting unit RUI is expressed with the following expression (2).

$$[ Math 2 ]$$

$${h}_{R}^{l} = σ ( {W}_{R}^{l} {h}_{D}^{l} ) \qquad ( 2 )$$

In the expression (2), a matrix W^{l}_{R} is a training parameter of the reporting unit RUI. A d-dimensional output vector h^{l}_{D} from the data unit DUI is reduced to an m-dimensional output vector h^{l}_{D} using the expression (2). σ is a sigmoid function.

A harmonizing unit HUL (2≤l≤L) is provided between the data unit DUI of the intermediate layer and the reallocation unit RAU per data unit DUl of the intermediate layer. The harmonizing unit HUL converts the number of dimensions of each piece of output data from the data unit DUl of the intermediate layer to the same size. Therefore, the reallocation unit RAU receives input of output data that is adjusted to the same number of dimensions using the harmonizing unit HUl.

That is, the harmonizing unit HUI receives input of the output vector h^{l}_{D} from the data unit DUl in the same layer and converts the number of dimensions of the output vector h^{l}_{D} to the same number of dimensions. A function h^{l}_{H} indicating the harmonizing unit HUL is expressed with the following expression (3).

$$[ Math 3 ]$$

$${h}_{H}^{l} = {f}_{H} ( {W}_{H}^{l} {h}_{D}^{l} ) \qquad ( 3 )$$

$$Where ,$$

$${W}_{H}^{l} ∈ {ℛ}^{{d}^{1} \times {d}^{l}} is a training parameter$$

In the expression (3), a matrix Why is a training parameter of the harmonizing unit HUl. With this, the d-dimensional output vector h^{l}_{D} from the data unit DUl is converted to an m-dimensional output vector h^{l}_{H}. m is a hyperparameter that determines the number of dimensions. d and m may have values different from d and m of the reporting unit RUl. f_{H} an activation function.

An attention unit AU calculates a weight α for each data unit DUl using the output vector h^{l}_{R} from each reporting unit RUl. A function a indicating the attention unit AU is expressed with the following expression (4).

[Math 4]

α=softmax(W_{A}h_{R}) (4)
Where, W_{A}∈ [symbol] ^{(L-1)×m} (m=r (L−1)) is a training parameter

In the expression (4), a matrix W_{A} is a training parameter of the attention unit AU. In the softmax function being one of the activation functions, a vector h_{R} with dimensions equal to a layer number L (L=4 in the example of the following expression (5)) is calculated. As indicated by the following expression (5), the vector h_{R} on the right-hand side of the expression (4) is a vector obtained by stacking h^{l}_{R} in a vertical direction.

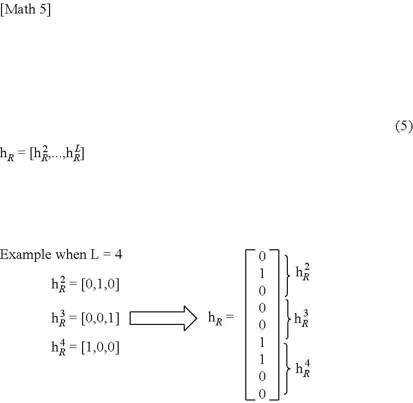

Therefore, the matrix W_{A} is a matrix with L rows and M columns (M is the number of elements in the vector h_{R}). By adopting the softmax function in the attention unit AU, each element (sum of all elements is 1) of the vector h_{R} of the layer number L represents the weight of the corresponding data unit DUl.

The reallocation unit RAU rearranges a feature vector of a certain feature space (image xₙ) to another feature space. Specifically, for example, as shown in FIG. 9, since the prediction model obtained from the feature vector group in the feature space SP1 can be a nonlinear prediction model, the reallocation unit RAU transfers the feature vector group to the feature space SP2 so that a linear prediction model can be obtained in the feature space SP2. A function h^{l}_{T} indicating the reallocation unit RAU is expressed with the following expression (6).

$$[ Math 6 ]$$

$${h}_{T}^{l} = {f}_{T} ( {h}_{H}^{l} , {x}_{n} ) \qquad ( 6 )$$

A Hadamard product of vectors, element addition, or the like can be used for a function f_{T}. In the present example, a Hadamard product is used (see the following expression (7)). In the expression (7), a Hadamard product of the output vector h^{l}_{H} from the harmonizing unit HUl and the feature vector xₙ is used.

$$[ Math 7 ]$$

$${h}_{T}^{l} = {h}_{H}^{l} ⊙ {x}_{n} \qquad ( 7 )$$

The unify unit UU integrates an output vector h^{l}_{T} from the reallocation unit RAU and an output vector α from the attention unit AU. That is, the unify unit UU weights the output vector h^{l}_{T} from the reallocation unit RAU with the output vector α from the attention unit AU. A function h_{U} indicating the unify unit UU is expressed with the following expression (8).

$$[ Math 8 ]$$

$${h}_{U} = {\sum }_{k = 1}^{L - 1} α [ k ] {h}_{T}^{k + 1} \qquad ( 8 )$$

In the expression (8), α [k] on the right-hand side indicates a k-th dimensional element (weight) of the output vector α in the expression (4).

The decision unit DCU determines a prediction value yₙ and outputs the prediction value yₙ to the output layer 1203. Specifically, the decision unit DCU obtains the prediction value yₙ by weighting an output vector h_{U} from the unify unit UU with a weight vector wₒ being one of the training parameters, and applying the output vector h_{U} to the sigmoid function σ, for example. A function yₙ indicating the decision unit DCU is expressed with the following expression (9). In the expression (9), t in wₒ^{t} signifies transposition.

$$[ Math 9 ]$$

$${y}_{n} = σ ( {w}_{O}^{t} {h}_{U} ) \qquad ( 9 )$$

The importance unit IU calculates an importance degree vector s^{l}ₙ indicating the importance degree of the features in each layer of the neural network, and outputs importance degree vector s^{l}ₙ to the output layer 1203. A function s^{l}ₙ indicating the importance unit IU is expressed with the following expression (10).

$$[ Math 10 ]$$

$${s}_{n}^{l} = α [ l ] {f}_{T} ( {w}_{O} , {h}_{H}^{l} ) \qquad ( 10 )$$

In the expression (10), α [l] on the right-hand side indicates an l-th dimensional element (weight) of the output vector α in an expression (12) described in Japanese Patent Laid-Open No 2020-194414. A Hadamard product of vectors, element addition, or the like can be used for the function f_{T}, similar to the expression (6). A Hadamard product may be used in the present embodiment. In the expression (10), the importance degree vector s^{l}ₙ is a Hadamard product of the weight vector wₒ and the output vector h^{l}_{H} from the harmonizing unit HUl. The importance degree vector sin is an importance degree in the layer l of the n-th feature vector (image) xₙ.

### Functional Configuration Example of Data Analysis Assistance Apparatus 100

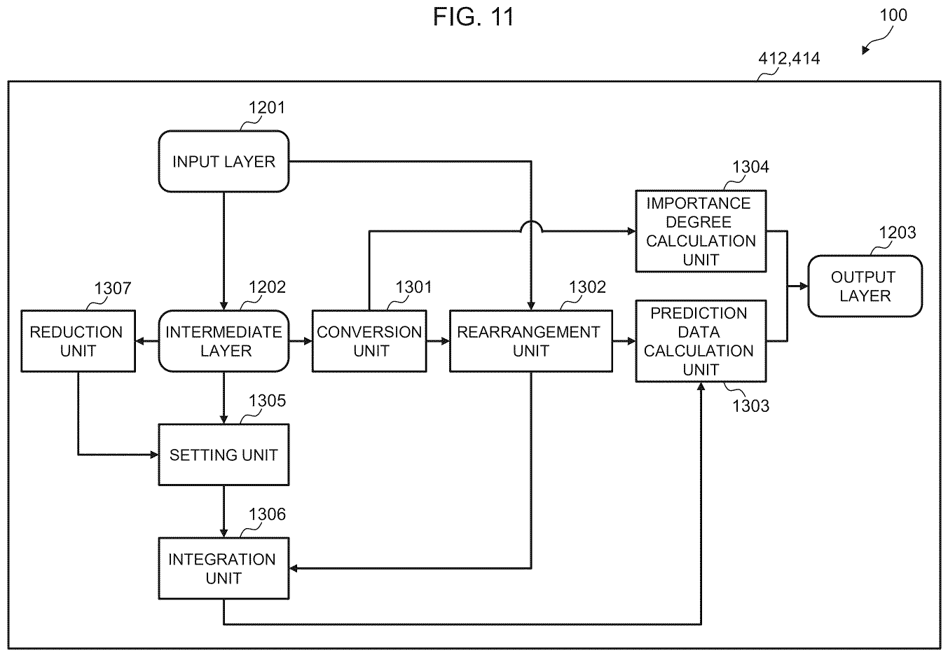

FIG. 11 is a block diagram showing a functional configuration example of part of the data analysis assistance apparatus 100. The data analysis assistance apparatus 100 includes the input layer 5201, an intermediate layer 1202, an output layer 1203, a conversion unit 1301, a rearrangement unit 1302, a prediction data calculation unit 1303, an importance degree calculation unit 1304, a setting unit 1305, an integration unit 1306, and a reduction unit 1307. The data analysis assistance apparatus 100 may include the training unit 412 and/or the prediction unit 414, which may be examples of an internal configuration of the training unit 412 and/or the prediction unit 414. The training unit 412 and/or the prediction unit 414 may be included in at least part of the above-described functions 120 to 124, or may be separate from the above-described functions 120 to 124.

As shown in the expression (3), the conversion unit 1301 reduces the dimension number d of the output vector h^{l}_{D} and outputs the converted output vector h^{l}_{H}, based on the output vector h^{l}_{D} from each data unit DUl (l≥2) of the intermediate layer and the matrix W^{l}_{H}. The conversion unit 1301 is the above-described harmonizing unit group HU.

As shown in the expressions (6) and (7), the rearrangement unit 1302 rearranges the feature vectors xₙ in the feature space SP1 to the feature space SP2, based on the converted output vector h^{l}_{H} from the conversion unit 1301 and the feature vectors xₙ in the feature space SP1 provided to the input layer 5201. The rearrangement unit 1302 is the above-described reallocation unit RAU.

As shown in the expression (9), the prediction data calculation unit 1303 calculates a prediction vector yₙ for the feature vectors xₙ in the feature space SP1, based on a rearrangement result h_{T}^{l} from the rearrangement unit 1302 and the weight vector wₒ. The prediction data calculation unit 1303 is the above-described decision unit DCU.

As shown in the expression (10), the importance degree calculation unit 1304 calculates the importance degree vector s^{l}ₙ of the feature vectors xₙ in the layer 1 in the intermediate layer 1202, based on the converted output vector h^{l}_{H} and the weight vector wₒ. The importance degree calculation unit 1304 is the above-described importance unit IU.

For example, regarding the image xₙ displaying an animal, an output vector h^{la}_{D} of a certain layer la is a feature indicating whether the facial contour is suitable for a cat and an output vector h^{lb}_{D} of a certain layer lb (≠la) is a feature indicating whether a shape of the ears is suitable for a cat. In this case, the user can explain which facial characteristics the data analysis assistance apparatus 100 has taken into consideration to determine that the animal in the image Xₙ is a cat, by referring to the corresponding importance degree vectors s^{la}ₙ and s^{lb}ₙ. For example, when the importance degree vector s^{la}ₙ is low but the importance degree vector s^{lb}ₙ is high, the user can explain that the data analysis assistance apparatus 100 has taken into consideration the shape of the ears to determine that the animal in the image xₙ is a cat. The calculated importance degree vector s^{l}ₙ is extracted by the importance degree calculation unit 415.

As shown in the expressions (4) and (5), the setting unit 1305 sets the weight α of the intermediate layer 1202, based on the output vector h^{l}_{D} from the intermediate layer 1202 and the matrix W_{A}. The setting unit 1305 is the above-described attention unit AU.

As shown in the expression (8), the integration unit 1306 integrates the rearrangement result h_{T}^{l} and the weight α set by the setting unit 1305. The integration unit 1306 is the above-described unify unit UU. In this case, the prediction data calculation unit 1303 calculates the prediction vector yₙ, based on an integration result hᵤ from the integration unit 1306 and the weight vector wₒ. The importance degree calculation unit 1304 calculates an importance degree vector sₙ^{l}, based on the weight α set by the setting unit 1305, the converted output vector h^{l}_{H}, and the weight vector wₒ.

As shown in the expression (2), the reduction unit 1307 reduces the dimension number d of the output vector h^{l}_{D} and outputs the reduced output vector h^{l}_{R}, based on the output vector h^{l}_{D} from the intermediate layer 1202 and the matrix W^{l}_{R}. The reduction unit 1307 is the above-described reporting unit group RU. In this case, the setting unit 1305 sets the weight α of the intermediate layer 1202, based on the reduced output vector h^{l}_{R} from the reduction unit 1307 and the matrix W_{A}.

When the training unit 412 is provided with the training data including the feature vectors xₙ in the feature space SP1 and the correct label tₙ for the prediction vector yₙ, the training unit 412 uses the prediction vector yₙ and the correct label tₙ to optimize the matrix W^{l}_{D} being a first training parameter, the matrix W^{l}_{H} being a second training parameter, the weight vector wₒ being a third training parameter, the matrix W_{A} being a fourth training parameter, and the matrix W^{l}_{R} being a fifth training parameter, such that cross-entropy between the correct label tₙ and the prediction value yₙ is minimized, for example.

The prediction unit 414 ultimately causes the prediction data calculation unit 1303 to calculate a prediction vector y′ₙ, by setting the optimized training parameters for the neural network 1200 and providing a feature vector x′ₙ as test data to the input layer 5201.

In this way, by rearranging the feature vectors xₙ being sample data in advance, it is possible to calculate the importance degree of each feature even if the neural network is multi-layered, and to accurately and efficiently realize ease of explanation per sample (feature vector xₙ). By rearranging the samples (feature vectors xₙ) in advance, it is possible to accurately calculate a prediction value with a low load during training and prediction, since a linear prediction model can be obtained.

The data analysis assistance apparatus 100 includes the conversion unit 1301, the rearrangement unit 1302, and the importance degree calculation unit 1304. Therefore, by rearranging the feature vectors (xₙ, x′ₙ) in advance, it is possible to accurately calculate a prediction value with a low load during training and prediction, since a linear prediction model can be obtained. It is possible to know what type of characteristics are present in the feature vectors (xₙ, x′ₙ) based on the importance degree per layer l from the importance degree calculation unit 1304. With this, it is possible to accurately and efficiently realize ease of explanation for the feature vectors (xₙ, x′ₙ) provided to the neural network as analysis targets.

Since the data analysis assistance apparatus 100 includes the prediction data calculation unit 1303, it is possible to accurately and efficiently realize ease of explanation as to why the prediction results (yₙ, y′ₙ) are obtained from the neural network as analysis targets with respect to the feature vectors (xₙ, x′ₙ).

Since the data analysis assistance apparatus 100 includes the setting unit 1305 and the integration unit 1306, the prediction data calculation unit 1303 can accurately calculate the prediction results based on the rearrangement result.

Since the data analysis assistance apparatus 100 includes the reduction unit 1307, it is possible to improve efficiency of the data analysis through dimension reduction.

The data analysis assistance apparatus 100 can construct a highly accurate prediction model by learning the training parameters.

Since the importance degree is determined in this way per feature, a selection unit 411 can select non-obvious features based on the importance degree.

1. A data analysis assistance apparatus comprising:
a feature vector generation unit configured to generate a feature vector for each of a plurality of samples, based on a data group relating to the plurality of samples;
a model generation unit configured to generate a linear model that predicts an evaluation value of predetermined circumstances, based on the feature vector of each of the plurality of samples and the evaluation value relating to the predetermined circumstances of a corresponding one of the plurality of the samples;
a clustering unit configured to perform clustering of the plurality of samples and generate clusters, based on coefficients of the linear model for each of the plurality of samples; and
an inverse analysis unit configured to perform inverse analysis of the feature vector for each of the generated clusters and identify data corresponding to the feature vector from the data group relating to the plurality of samples.

2. The data analysis assistance apparatus according to claim 1, further comprising an importance degree calculation unit configured to calculate an importance degree of the feature vector in the linear model, wherein
the inverse analysis unit performs inverse analysis of the feature vector with the importance degree equal to or larger than a threshold value.

3. The data analysis assistance apparatus according to claim 1, wherein the data relating to the plurality of samples is data relating to a medicine.

4. The data analysis assistance apparatus according to claim 1, further comprising an output unit configured to output information about the data corresponding to the identified feature vector.

5. A data analysis assistance method comprising:
processing, by an information processing apparatus, of generating a feature vector for each of a plurality of samples, based on a data group relating to the plurality of samples;
processing, by the information processing apparatus, of generating a linear model that predicts an evaluation value of predetermined circumstances, based on the feature vector of each of the plurality of samples and the evaluation value relating to the predetermined circumstances of a corresponding one of the plurality of the samples;
processing, by the information processing apparatus, of performing clustering of the plurality of samples and generating clusters, based on coefficients of the linear model for each of the plurality of samples; and
processing, by the information processing apparatus, of performing inverse analysis of the feature vector for each of the generated clusters and identifying data corresponding to the feature vector from the data group relating to the plurality of samples.
